(19) United States
(12) Patent Application Publication
(10) Pub. No.: US 2005/0053563 A1
(43) Pub. Date: Mar. 10, 2005

(54) Stable compositions containing ethanolamine derivatives and glucosides

(76) Inventors: Patricia Manissier, Levallois Perret (FR); Muriel Morelli, Acquiny (FR); Arnaud Buronfosse, Paris (FR); Aude Seigneurin, Boulogne-Billancourt (FR); Agnes Le Fur, Louviers (FR)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/491,045

(22) PCT Filed: Sep. 27, 2002
(86) PCT No.: PCT/EP02/11058

(30) Foreign Application Priority Data
Sep. 27, 2001 (EP) 01402484.8

(51) Int. Cl. A61K 7/06; A61K 7/11

(52) U.S. Cl. 424/70.13

(57) ABSTRACT

This invention relates to stable compositions comprising an ethanolamine derivative and one or more glucosides (in particular melibiose and lactose). It further relates to the use of such compositions in cosmetic preparations, in particular in anti-aging formulations. The invention further relates to the use of such compositions to promote normal human dermal fibroblasts growth and to stimulate collagen synthesis. It further concerns a process for preparing such compositions.

### BRIEF DESCRIPTION OF THE INVENTION

This invention relates to stable compositions comprising an ethanolamine derivative and one or more glucosides (in particular melibiose and lactose). It further relates to the use of such compositions in cosmetic preparations, in particular in anti-aging formulations. The invention further relates to the use of such compositions to promote normal human dermal fibroblasts growth and to stimulate collagen synthesis. It further concerns a process for preparing such compositions.

### BACKGROUND OF THE INVENTION

Skin is composed of three integrated layers: the epidermis, the dermis and the hypodermis. The thickness of the epidermis and the dermis varies over different parts of the body. The epidermis also grows into fingernails, toenails and hair. The epidermis is principally composed of three types of cells: keratinocytes (90% of epidermal cells), the melanocytes (2-8% of epidermal cells) and Langerhans' cells.

The dermis, or true skin, is thick, sturdy, rich in nerves and blood vessels and in perspiratory glands. It also functions to shield and repair injured tissue. The dermis consists mostly of collagen, which originates from cells called fibroblasts, elastin structural glycoproteins and proteoglycans. Collagen fibers, which represent 70% of the dry weight of the dermis, form a supporting mesh responsible for skin's mechanical characteristics such a strength, texture and resilience. Other cells such as macrophages and leukocytes are also present in the dermis layer.

The hypodermis, joined to the bottom of the dermis, is the deepest layer of the skin. It contains so-called ‘adipocytes’ which produce lipids that build the fatty layer in the subcutaneous tissue. This layer functions to protect muscles, bones and inner organs against shocks, and to act as an insulator and source of energy during lean times.

As a first sign of aging, skin becomes less elastic and develops fine lines and wrinkles, which are the direct result of deterioration of the supporting dermis layer. In fact the skin's ability to replace damaged collagen diminishes and more and more disconnections and irregularities develop in the collagen network. Further phenomena associated with skin aging are the appearance of pigment marks, skin thinning and skin sagging. Many factors contribute to accelerated collagen damage. These include sun exposure, the presence of free radicals, some age-related hormonal changes, and smoking.

Aging of the skin is attributed to two causal factors. On the one hand there is chronological or intrinsic aging while on the other there is extrinsic aging, or aging due to environmental factors. Amongst the latter there can be mentioned photo-aging, which is the damage caused to the skin due to direct or indirect effects of the ultraviolet spectrum of sunlight.

A number of treatments have been developed that have proved out to be more or less effective in combating the effects of skin aging. Cosmetic products have been introduced which contain vitamins or vitamin derivatives, in particular vitamin A or its derivatives (retinoids), vitamin C, alpha-hydroxy acids or plant extracts. These products, when applied regularly during longer periods of time, reduce the number of wrinkles and fine wrinkles. Collagen implants on the other hand can disguise expression lines around the eyes and mouth. Dermabrasion and chemical peels are applied to remove the top layer of damaged skin. Carbon dioxide laser resurfacing is applied to remove fine wrinkles and improve scars.

A particular pathway used in the treatment of the effects of skin aging is by stimulation of dermal human fibroblasts and collagen formation. Agents possessing these properties for example are L-ascorbic acid and in particular retinol.

Other agents that have been described to be useful to treat the effects of skin aging are the ethanolamine derivatives. U.S. Pat. No. 5,554,647 describes a method of treating aging skin and subcutaneous muscles comprising the use of an acetylcholine precursor such as dimethylaminoethanol (DMAE) in an amount effective to produce increased muscle tone.

U.S. Pat. No. 5,643,586 describes the topical treatment of subcutaneous muscle and overlying cutaneous tissue by applying a composition comprising a catecholamine precursor which in particular is tyrosine, phenylalanine or a mixture thereof preferably in combination with an acetylcholine precursor such as dimethylaminoethanol.

Certain glucosides have been described to have beneficial effect on cell metabolism and on extracellular matrix synthesis in dermal fibroblasts.

Although these methods and the products used therein have been applied with varying degrees of success, there nevertheless remains room for improvement. In particular there still is a need for new formulations that are more effective in combating the effects of the skin aging process and in particular promote skin cell renewal and extracellular matrix production.

One way to achieve this goal would be to combine certain active ingredients in one formulation and more specifically a formulation containing both an aminoethanol and a glucoside would seem attractive. However it has been found that compositions containing ethanolamines and glucosides, and in particular compositions wherein the glucosides are melibiose and/or lactose, are not stable. A number of undesired side or decomposition reactions were found to take place, including for example Maillard type of reactions. This resulted in the presence of undesired side or decomposition products in the compositions. Some of the side or decomposition products moreover caused an undesired coloration of such compositions.

Compositions containing ethanolamines and in particular those containing dimethylaminoethanol should have a pH that is sufficiently high, in particular equal or above pH 6 or higher, in order for the ethanolamine to be effective. Ethanolamines are typically used in appropriate salt forms, in particular as alpha hydroxy acid salt forms such as citrates or glycolates, or which is preferred, as mixed salts. These tend to lower the pH. In that instance, an obvious way to achieve this a pH of 6 or higher is to add basic components to the composition, e.g. suitable metal hydroxides.

Too high a pH on the other hand has been found to result in polymerisation of the glucoside which is particularly the case when the pH is above pH 7. The pH should not be too high for reasons of skin compatibility the pH should preferably not exceed pH 7.

Therefore the compositions of an ethanolamine and a glucoside should preferably have a pH which is in the range of about pH 6 to about pH 7.

It was further found that formulations containing a combination of a glucoside and an ethanolamine resulted in an undesired decrease of the pH during storage of the product. This resulted in a shortening of the product's shelf life below acceptable levels.

It is therefore an object of the present invention to provide stable compositions, in particular such compositions having a pH, which is in the range of 6 to 7. It is a further object to provide a process for manufacturing such compositions. It is another object to provide compositions containing an ethanolamine and a glucoside having a sufficiently long shelf life.

These objects are attained by the compositions and processes according to the present invention which will be described hereinafter in more detail.

### SUMMARY OF THE INVENTION

The present invention is directed to a stable composition comprising at least one ethanolamine derivative of formula I, or a topically acceptable salt thereof: 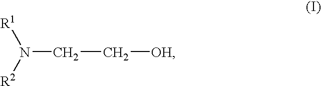
and at least one glucoside.

In formula I, R¹ and R² independently represent hydrogen, C₃₋₆ cycloalkyl or C₁₋₆ alkyl, optionally substituted with hydroxy, methoxy, oxo or formyl.

Preferably R¹ and R² independently represent C₁₋₄ alkyl.

The most preferred ethanolamine of formula I is dimethylaminoethanol (DMAE), also referred to as deanol. Preferred salts are alpha hydroxy acid salts. Most preferred salts are glycolic and citric acid salts, or combined salts.

The invention further relates to a stable composition, as defined above, having a pH in the range of about 6 to about 7. In a particular aspect the invention relates to a composition, as defined above, additionally containing an amount of a metal hydroxide, such that the pH does not exceed 7. In a further particular aspect the invention relates to a composition, as defined above, additionally containing an amount of a buffer effective in the range of pH 6 and pH 7.

Particular glucosides are melibiose and lactose.

The invention further is concerned with a stable topical formulation comprising a composition as defined herein and further ingredients. The topical formulation can be for dermatological use, but in particular is for cosmetic use.

In another aspect the present invention provides a process for preparing a composition as defined hereinabove or hereinafter comprising the steps of:
(1) preparing a first aqueous phase comprising the glucoside, optionally in admixture with other components while keeping the pH below 7;
(2) preparing a second aqueous phase comprising an ethanolamine of formula I and a suitable base, optionally in admixture with other components;
(3) adding the second mixture to the first mixture in a controlled manner such that the pH of the mixture stays under pH 7.

In particular embodiments of this process the following apply:
(a) the pH of the second aqueous phase is above pH 6 and in particular is above pH 7 by adding an appropriate amount of base; and/or
(b) the pH of the mixture obtained in step (3) is kept at a pH in the range of about pH 6 to pH 7

In a further aspect there is provided a stable composition comprising at least one ethanolamine derivative of formula I, or a topically acceptable salt thereof, wherein the ethanolamine of formula I is as defined hereinabove or hereinafter, and at least one glucoside, said composition being obtained or obtainable by a process as defined hereinabove or hereinafter.

In another aspect the invention provides the use of a composition as defined herein for manufacturing a topical or in particular a cosmetic formulation. The topical or cosmetic formulations in particular are for combating or treating the effects of skin aging.

In a further aspect the invention provides the use, and in particular the cosmetic use, of a composition as defined herein, or of a topical formulation as defined herein, for combating or treating the effects associated with skin aging.

Or, alternatively, the invention concerns a method, and in particular a cosmetic method, of combating or treating the effects of skin aging, which method or cosmetic method comprises applying to the affected skin area an amount of a composition or a topical formulation as defined herein, said amount being effective to treat said effects of skin aging.

Still another aspect of this invention comprises a cosmetic method for the improvement of the external appearance of an individual, said method comprising applying a composition or a topical composition as defined herein to affected skin areas.

### DETAILED DESCRIPTION OF THE INVENTION

The compositions of the present invention contain an ethanolamine of formula I as defined above. As used herein C₃₋₆ cycloalkyl refers to a cyclic cycloalkyl radical that preferably is saturated such as for example cyclopropyl, cyclobutyl, cyclopentyl and cyclohexyl. C₁₋₆ alkyl refers to straight and branch chained hydrocarbon radicals which preferably are saturated and have from 1 to 6 carbon atoms such as, for example, methyl, ethyl, n.propyl, iso-propyl, n.butyl, iso-butyl, t.butyl, n.pentyl, iso-pentyl, 2-methylbutyl, 2,2-dimethylpropyl, n-hexyl, 2-methylpentyl, 2,2-dimethylbutyl and the like. C₁₋₄ alkyl refers to the same group of radicals with those having 5 or 6 carbon atoms being excluded.

The ethanolamines of formula I can be prepared according to art-known procedures, e.g. by alkylating ethanolamine.

The ethanolamines of formula I can be used in its basic form or can be used as an appropriate topically acceptable salt, the latter referring to acid-addition salt forms that are biologically acceptable for the skin and/or mucous membranes. Biologically acceptable in particular refers to lack of toxicity and of adverse effects such as irritation, allergic reactions and the like.

Suitable topically acceptable salts are those that are obtained by treating the base form of the ethanolamine of formula I with an appropriate acid. Suitable acids for use in the preparation of the ethanolamine salts according to the invention include any inorganic or, which is preferred, organic acid known to be useful in skin care compositions. These include acids such as, for example, inorganic acids such as hydrohalic acids, e.g. hydrochloric or hydrobromic acid; boric, sulfuric, nitric, phosphoric and the like acids; or organic acids such as, for example, acetic, propanoic, hydroxyacetic (or glycolic), lactic, pyruvic, oxalic, malonic, succinic, maleic, fumaric, malic, tartaric, citric, methanesulfonic, ethanesulfonic, benzenesulfonic, p-toluenesulfonic, cyclamic, salicylic, p-aminosalicylic, ascorbic and the like acids.

In a preferred embodiment, at least one of the acids is an alpha hydroxy acid, such as taught for example in U.S. Pat. No. 5,856,357, the disclosure of which is hereby incorporated by reference. Particularly preferred is a mixture of at least two of glycolic acid, malic acid and citric acids. In a most preferred embodiment, the acid is a combination of glycolic acid and either malic or citric acid. In situations where pH stability is a particular concern, e.g., long term storage, a particularly preferred embodiment is when the acid is a mixture of citric and glycolic acid. Preferably, the ratio of malic or citric acid to glycolic acid ranges from about 1:1 to about 1:5, more preferably, from about 1:1 to about 1:3.

Another compound which is advantageously present in the compositions of this invention is tyrosine. Tyrosine may be present in the compositions of this invention in the amount of from about 0.01 to about 5%, more preferably from about 0.04 to about 3% by weight and most preferably about 0.5% by weight, based on the total composition.

The compositions of the invention are stable which means that they can be stored at standard conditions during periods of time which are common in the trade, in particular in the trade of cosmetics. In particular the active ingredients of formula I and the glucosides will remain chemically unaffected, in particular without showing the undesired deteriorating reactions mentioned above. The compositions of the invention and the topical formulations derived therefrom remain intact during standard shelf-life periods at ambient temperature, e.g. longer than 2 years at a temperature of about 25° C.

The compositions of the invention further contain at least one glucoside, which in particular is melibiose, lactose or a mixture thereof.

In the compositions of the present invention the w/w ratio of the ethanolamine of formula I to the glucoside(s) may vary but in particular is in the range of about 2:1 to 1:5, more in particular in the range of about 2:1 to 1:3, preferably from about 1:1 to 1:2, more preferably the w/w ratio is about 1:1.5.

The ethanolamines of formula I, and more specifically dimethylethanolamine, can be used in the compositions or formulations for topical application at concentrations which are in the range of 0.001% to 10%, preferably in the range of 0.1% to 5% more preferably from 0.5 to 5%. The glucoside(s), and more specifically melibiose and/or lactose, may be used in the composition for topical application at concentrations between 0.1% to 20% and preferably between 0.5 to 10%. The percentages mentioned herein refer to w/w percentages of the component to the total weight of the end composition.

The compositions of the invention are made by a specific process. According to this process, a first aqueous phase mixture is made by mixing the glucoside(s) with water optionally with the other components while keeping the pH below 7. In a particular execution of the process, the pH of the first aqueous phase is kept above ph 5, or more in particular above pH 6. The optional other components in particular are neutral or acidic, but also basic components can be added as long as the pH remains below pH 7. Basic components may include suitable bases, such as for example, basic inorganic hydroxides such as alkali or earth alkaline metal hydroxides, preferably sodium or potassium hydroxide; or carbonates such as the alkali metal carbonates, e.g. sodium carbonate, or mixtures thereof. However no amino components and in particular ethanolamine components should be present in or added to the first phase.

The other components that are added in the first aqueous phase will mainly depend on the desired form of the composition. For example a thickener may be added in case of a gel. In case of emulsions an oily phase will be prepared and the oily phase may be emulsified into the first aqueous phase. In that instance the first aqueous phase may contain suitable emulsifiers, which may be neutral, amphoteric, basic or acidic. In the instance where these components are acidic, e.g. when using an acidic thickener or emulsifier, it may be recommendable to increase the pH by adding an appropriate amount of base, e.g. by adding sodium or potassium hydroxide, however while keeping the pH below pH 7.

In order to lower or keep the pH of the first aqueous phase below pH 7 suitable acids, in particular acids that are skin-compatible may be added. Suitable acids may be organic acids, in particular acids that have buffering capacity in the preferred pH range of pH 6 to pH 7. Of particular interest are the alpha hydroxy acids such as those, which are used to make the salt forms of ethanolamines.

However, one or more of the other components may be acidic in nature and in that instance no further acids need to be added. In the instance where the pH of the first aqueous phase is kept in the range of pH 5 to pH 7 or pH 6 to pH 7, it may be necessary to add suitable basic components, in particular skin-compatible basic components. More specifically this will be the case where one or more of the other components are acidic. These basic components will be other than ammonia or ammonia derivatives, including amines. Preferred are the alkali metal or alkaline earth metal hydroxides or carbonates, in particular the hydroxides.

In certain embodiments particular acids or acidic salts may be added to the first aqueous phase that are have buffering capacity in the preferred pH range of pH 6 to pH 7. Examples are the following acids or their acidic salts: phosphoric acid, tartaric acid, malic acid, oxalic acid and the like acids, and preferably alpha hydroxy acids such as, for example, citric and glycolic acid.

In a preferred embodiment the first aqueous phase is made by first mixing any acidic components with water and subsequently neutralizing the mixture to a pH, which is below pH 7, and preferably is above pH 6, whereupon the glucoside(s) is or are added. Acidic components can be thickeners, emulsifiers and acids such as citric or glycolic acid, or mixtures thereof.

A second aqueous phase is made comprising the ethanolamine of formula I and further, optionally, a suitable base. Suitable bases comprise the same suitable bases as mentioned above in respect to the preparation of the first aqueous phase, and in particular are an alkali metal hydroxide such as sodium or potassium hydroxide. Also in this instance ammonia or ammonia derivatives should be avoided.

In those instances where the process is conducted with a first aqueous phase containing a suitable base, the second aqueous phase may also contain amounts of the same or other suitable base or bases, preferably however in smaller amounts. Hence in that instance both phases contain suitable base, which may be the same or different. In certain embodiments however, where the first phase contains a suitable base, the second phase does not contain the same or other suitable bases.

The pH of the second aqueous phase is selected such that it is above pH 6 and in particular is above pH 7. Although in principle the pH of the second aqueous phase does not need to be limited, it is recommendable to have it not too high, e.g. below pH 10, or below pH 9, or even below pH 8. This may be prompted, for example, by the presence of specific additional components in the second aqueous phase that may be sensitive to environments having higher pH values.

In case base is added to the second phase, the ethanolamine may first be mixed with water after which the base is added, or vice versa. The base preferably is an alkali metal hydroxide which may be added in solid form or preferably dissolved in water.

The second aqueous phase may also contain acidic components such as organic acids such as, for example, citric and glycolic acid. Of particular interest are organic acids or acidic salts that form a buffer in the preferred pH range of pH 6 to pH 7.

In a particular embodiment an aqueous mixture is made of the ethanolamine of formula I and an appropriate amount of base, in particular sodium or potassium hydroxide, is added. Appropriate amounts are selected in function of the acidic components that are present in the aqueous phases.

In an alternative embodiment the ethanolamine of formula I is mixed with water and an acid is added which preferably is an organic acid such as an alpha hydroxy acid, for example citric acid and glycolic acid, and an appropriate amount of base, in particular sodium or potassium hydroxide. Preferably those acids or acidic salts are used which have buffering capacity in the preferred pH range of pH 6 to pH 7.

The first and second aqueous phases can be made in any given sequence.

The second mixture is subsequently added to the first mixture while controlling the pH in such manner that it stays under pH 7. Preferably the pH of the end mixture is kept in the range of about pH 6 to about pH 7.

An essential feature of the present invention comprises the fact that by controlling the process by which the formulation is made, it becomes possible to produce a composition that contains a glucoside and an ethanolamine that is stable and that remains stable all along the product's shelf life. The compositions made through the process do not show discoloration and lack side or decomposition products. This equally applies to any topical formulations derived from these compositions.

The compositions and formulations according to the invention, made according to the above-described process, contain salts of one ore more suitable organic acids. These salts are derived from the bases and salts that have been used to control the pH within the ranges set forth hereinabove. In particular these salts are derived from a suitable organic acid or organic acid mixture. The latter in particular are organic acids or acidic salts having buffering capacity in the preferred pH range of pH 6 to pH 7. More specifically these are alpha hydroxy acids such as the ones mentioned herein. The cation of these salts is derived from a suitable base, which is other than ammonia or an ammonia derivative (or other than an amine or an amine derivative). Of special interest are alkali metal or alkaline earth metal salts. Preferably, the compositions and formulations of the invention contain alkali metal salts of alpha hydroxy acids, more preferably of glycolic and citric acid. Of particular interest are compositions or formulations according to the invention further containing sodium or potassium salts of glycolic and/or citric acid. In particular embodiments the latter acids are present in the same weight ratios as cited above in relation to the ethanolamine salts. The nature and quantity of such salt mixtures will be selected such that it is in the range of pH 6 to pH 7. This can be one by calculation or by experimentation or both.

The compositions and formulations subject of the present invention are useful to combat or to treat the effects of skin aging. The effects of skin aging comprise typical features associated with aging skin such as, for example, the appearance of fine lines, fine wrinkling, wrinkling, loss of skin firmness, skin tightening and suppleness. The effects of skin aging may be due to aging as such but also to aging of the skin caused by external factors such as exposure to environmental factors such as sunlight, wind, atmospheric poluants and the like, or a combination of these factors.

As mentioned above, ethanolamine derivatives are known to stimulate the proliferation of dermal cells and in particular the proliferation of fibroblasts, and the production of collagen. This is also the case with certain glucosides, in particular with lactose and/or melibiose.

Compositions containing the ethanolamines of formula I and glucosides, as well as topical formulations containing these compositions are useful for the treatment of the effects of skin aging. These compositions and formulations act through the stimulation of collagen production and promotion of fibroblast growth. Additionally the effect of both agents is potentiated by the other so that both agents act synergistically.

The topical formulations according to the invention may be in the form of a solution, a lotion, either a hydrophilic lotion or an emulsion-based lotion, an ointment, a cream or a gel. The formulations may also be, for example, in the form of oil-in-water, water-in-oil or multiple emulsions, foaming products or in liposome form.

Preferred formulations are gel and cream based formulations. Of particular interest are formulations based on oil-in-water emulsions.

In the latter instance an oily phase, containing the oil-soluble components, is made separately which is added to any of the aqueous phases containing suitable emulsifiers. Preferably the first aqueous phase is made containing one or more suitable emulsifiers. The oily phase is made and added to the first phase while building an emulsion. Subsequently the second aqueous phase is added. In the instance where the first or oily phase contains solid or semi-solid components, it is recommendable to heat the phase or phases and to conduct the emulsifying process at elevated temperature.

All the topical formulations as described herein can be applied on the skin by means of wipes.

The topical formulations according to the invention can further include one or more of a variety of additional ingredients commonly found in skin care compositions, such as for example, emollients, skin conditioning agents, emulsifying agents, humectants, preservatives, antioxidants, active ingredients, perfumes, chelating agents, dyes, opacifying agents, etc., provided that they are physically and chemically compatible with the other components of the composition. Noteably useful is the incorporation of vitamin A and vitamin A derivatives, including but not restricted to retinol, retinyl palmitate, retinoic acid, retinal, and retinyl propionate.

Examples of other active agents which may be incorporated comprise anti-microbials, e.g. anti-bacterials and antifungals, anti-inflammatory agents, anti-irritating compounds, anti-itching agents, moisturising agents, skin caring ingredients, plant extracts, vitamins, and the like. Also included are sunscreen actives which may be inorganic or organic in nature.

Examples of suitable preservatives for use in the compositions or formulations of the invention include the C₁-C₄ alkyl parabens and phenoxyethanol. Generally, the preservative is present in an amount ranging from about 0.5 to about 2.0, preferably about 1.0 to about 1.5, weight percent based on the total composition. In a preferred embodiment, the preservative is mixture of from about 0.2 to about 0.5 weight percent methylparaben, from about 0.2 to about 5.0 weight percent propylparaben and from about 0.05 to about 0.10 weight percent butylparaben. A particularly preferred commercially available preservative that may be used in the skin care composition according to this invention is Phenonip™ which is a practically colorless, viscous, liquid mixture of phenoxyethanol, methylparaben, ethylparaben, propylparaben, and butylparaben available from Nipa Laboratories, Inc.

Preferably, antioxidants should be present in the compositions or formulations according to the invention. Suitable antioxidants include butylated hydroxy toluene (BHT), ascorbyl palmitate, butylated hydroanisole (BHA), phenyl-α-naphthylamine, hydroquinone, propyl gallate, nordihydroquiaretic acid, vitamin E or derivatives of vitamin E, vitamin C and derivatives thereof, calcium pantothenic, green tea extracts and mixed polyphenosls, and mixtures thereof of the above. When utilized the antioxidant can be present in an amount ranging from about 0.02 to about 0.5% by weight, more preferably from about 0.002 to about 0.1% by weight of the total composition.

Emollients which can be included in the compositions or formulations of the invention function by their ability to remain on the skin surface or in the stratum corneum to act as lubricants, to reduce flaking, and to improve the skin appearance. Typical emollients include fatty esters, fatty alcohols, mineral oil, polyether siloxane copolymers and the like. Examples of suitable emollients include, but are not limited to, polypropylene glycol (“PPG”)-15 stearyl ether, PPG-10 cetyl ether, steareth-10, oleth-8, PPG-4 lauryl ether, vitamin E acetate, PEG-7 glyceryl cocoate, lanolin, cetyl alcohol, octyl hydroxystearate, dimethicone, and combinations thereof. Cetyl alcohol, octyl hydroxystearate, dimethicone, and combinations thereof are preferred. When utilized, the emollient can be present in an amount from about 0.01 to about 5, preferably from about 1 to about 4 percent by weight based on the total composition.

Polyhydric alcohols can be utilized as humectants in the compositions or formulations of the invention. The humectants aid in increasing the effectiveness of the emollient, reduce scaling, stimulate removal of built-up scale and improve skin feel. Suitable polyhydric alcohols include, but are not limited to, glycerol (also known as glycerin), polyalkylene glycols, alkylene polyols and their derivatives, including butylene glycol, propylene glycol, dipropylene glycol, polypropylene glycol, polyethylene glycol and derivatives thereof, sorbitol, hydroxypropyl sorbitol, hexylene glycol, 1,3-dibutylene glycol, 1,2,6,-hexanetriol, ethoxylated glycerol, propoxylated glycerol and mixtures thereof. Glycerin is preferred. When utilized, the humectant is present in an amount from about 0.1 to about 5, preferably from about 1 to about 3 percent by weight, based on the total weight of the composition.

The topical formulations of the invention have an improved effect on skin damage due to aging. The first results may be obtained after four weeks of treatment with the compositions and are exerted deep down. These effects comprise a reduction in the number and depth of wrinkles and small wrinkles, a firming and tightening of the skin and providing a more youthful and smooth aspect of the skin, in particular of facial skin.

The topical formulations of the invention may be applied at any time of the day but preferably are applied in the morning and/or evening. They may be applied on those parts of the body where skin aging is prominent, i.e. on the face, the body or the hands.

The topical formulations of the invention are particularly appropriate for treating the areas around the eyes and the lips, which are very fragile and are highly susceptible to the appearance of wrinkles and loss of firmness of the skin. Compositions according to the invention are very well tolerated in less sensitive area, here their anti-aging activity is exerted from four weeks of application onwards. They make possible to reduce visibly the number of wrinkles and eye marks; they firm up the skin around the eyes and mouth which is particularly sensitive.

The following example is meant to illustrate the invention and not to limit it thereto. All percentages are by weight (w/w).

### EXAMPLE

| Ingredients | % (w/w) |
|---|---|
| First Aqueous Phase | |
| Aqua | 47.912 |
| Acrylates, C 10/30 Alkyl Acrylates | 0.500 |
| Crosspolymer mixture | |
| Aqua, (32%), Glyceryl Polymethacrylate | 5.000 |
| (67%), Propylene Glycol(1%) mixture | |
| Cetyl Phosphate | 1.880 |
| Aqua | 1.000 |
| Sodium Hydroxide | 0.202 |
| Melibiose | 0.500 |
| Lactose | 4.500 |
| L-Tyrosine | 0.500 |
| Lipid Phase | |
| Cetearyl Octanoate(90%), Isopropyl | 8.000 |
| Myristate(10%) mixture | |
| Cetyl Palmitate | 4.000 |
| Cetyl Alcohol | 1.500 |
| Dimethicone | 0.500 |
| Polyglyceryl 3 Diisostearate | 1.000 |
| Tocopheryl Acetate | 1.000 |
| Phenoxyethanol | 0.500 |
| Methylparaben | 0.200 |
| Propylparaben | 0.060 |
| Cetearyl Isononanoate | 6.000 |
| Second Aqueous Phase | |
| Aqua | 9.000 |
| Citric Acid | 0.510 |
| Glycolic Acid(70%), Aqua(30%) mixture | 2.186 |
| Dimethyl ethanolamine | 3.000 |
| Perfume | 0.550 |

The ingredients of the First Aqueous Phase are mixed in the sequence they are listed. Subsequently the Lipid Phase is made by mixing the lipid components in the sequence they are listed at a temperature of about 80° C.

The first Aqueous Phase is heated to about 80° C. and subsequently the lipid phase is added while stirring, thus forming an emulsion. The thus formed emulsion is allowed to cool whereupon the Second Aqueous Phase, which is prepared by mixing the ingredients in the sequence as listed, is added.

1. A stable composition comprising at least one ethanolamine derivative of formula I, or a topically acceptable acceptable salt thereof: 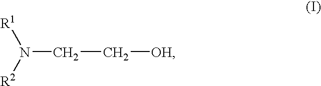
and at least one glucoside, wherein in formula (I) R¹ and R² independently represent hydrogen, C₃₋₆ cycloalkyl or C₁₋₆ alkyl, optionally substituted with hydroxy, methoxy, oxo or formyl.

2. A composition according to claim 1 wherein R¹ and R² independently represent C₁₋₄ alkyl.

3. A composition according to claim 1 wherein the ethanolamine of formula I is dimethylaminoethanol (DMAE).

4. A composition according to claim 1 wherein the composition has a pH in the range of about 6 to about 7.

5. A composition according to claims 1-4 wherein the glucosides are melibiose and lactose.

6. A stable topical formulation comprising a composition as claimed in claim 1 and further ingredients.

7. A process for preparing a composition as claimed in claim 1 further comprising a buffer that maintains the pH in the range of pH 6-pH 7, said method comprising the steps of:
(1) preparing a first aqueous phase comprising the glucoside, optionally in admixture with other components while keeping the pH below 7;
(2) preparing a second aqueous phase comprising the ethanolamine of formula I and a suitable base, optionally in admixture with other components; and
(3) adding the second mixture to the first mixture in a controlled manner such that the pH of the mixture stays under pH 7.

8. A process according to claim 7 wherein:
(a) the pH of the second aqueous phase is above pH 6 and in particular is above pH 7 by adding an appropriate amount of base; and/or
(b) the pH of the mixture obtained in step (3) is kept at a pH in the range of about pH 6 to pH 7.

9. A stable composition comprising at least one ethanolamine derivative of formula I, or a topically acceptable acceptable salt thereof, wherein the ethanolamine of formula I is as defined in claim 1, and at least one glucoside, obtained or obtainable by a process as defined in claim 7.

10. (Canceled)

11. (Canceled)

12. A method of manufacturing a topical composition, said method comprising the addition of a composition as claimed in claim 1.

13. A method for combating or treating the effects associated with the aging of skin, said method comprising the topical application of a composition as claimed in claim 1.
